(19) United States
(12) Patent Application Publication
(10) Pub. No.: US 2013/0018451 A1
(43) Pub. Date: Jan. 17, 2013

(54) Delivery System

(71) Applicants: Gerald Grabowski, Plymouth, MN (US); Tim Ramos, Blaine, MN (US); Jeremy Crawford, Woodbury, MN (US); Keith Anderson, Minneapolis, MN (US)

(72) Inventors: Gerald Grabowski, Plymouth, MN (US); Tim Ramos, Blaine, MN (US); Jeremy Crawford, Woodbury, MN (US); Keith Anderson, Minneapolis, MN (US)

(73) Assignee: BOSTON SCIENTIFIC SCIMED, INC., Maple Grove, MN (US)

(21) Appl. No.: 13/545,541

(22) Filed: Jul. 10, 2012

Related U.S. Application Data
(60) Provisional application No. 61/506,863, filed on Jul. 12, 2011.

(51) Int. Cl. A61F 2/84

(52) U.S. Cl. 623/1.12; 623/1.11

(57) ABSTRACT

Medical systems and devices are described. In one example, a system for delivering an implantable endoprosthesis into a body vessel is described. The system includes a handle assembly having a housing, a rotatable member, a first pulley engaged to the rotatable member, a second pulley rotatably engaged to the housing, and a belt extending between the first pulley and the second pulley, the belt engaged to the first pulley. The system further includes an inner member, an outer sheath secured to the belt and disposed about at least a portion of the inner member. The system excludes a rack.

### CROSS-REFERENCE TO RELATED APPLICATIONS

This application claims priority to U.S. Provisional Application No. 61/506,863, filed Jul. 12, 2011, the entire contents of which are herein incorporated by reference.

### TECHNICAL FIELD

The disclosure relates to medical devices and, more particularly, to medical devices that deliver implantable endoprostheses into a body vessel.

### BACKGROUND

Devices are known for delivering implantable endoprostheses, such as stents, into a body vessel. Devices of this kind often include a proximal portion that remains external to the body vessel during use and a distal portion that is inserted into the body vessel (e.g., through an incision). The proximal portion typically provides for manipulation of the device during use. The distal portion often includes an outer member slidably positioned about an inner member with an endoprosthesis disposed therebetween. Generally, the distal portion of the device is advanced through the body vessel to a treatment site (e.g., a stenosis or aneurysm). The outer member can then be refracted to allow the endoprosthesis to expand to engage a wall of the body vessel at the treatment site. Thereafter, the device is removed leaving the endoprosthesis engaged with the body vessel.

### SUMMARY

In general, this disclosure describes systems and techniques for delivering an implantable endoprosthesis into a body lumen of a patient. More particularly, this disclosure describes a delivery system that utilizes a belt and pulley system, which allows the entire deployment mechanism to reside in a handle of the system, thus significantly shortening the length of the handle.

In one example, the disclosure is directed to a system for delivering an implantable endoprosthesis into a body vessel. The system comprises a handle assembly that comprises a housing, a rotatable member, a first pulley engaged to the rotatable member, a second pulley rotatably engaged to the housing, and a belt extending between the first pulley and the second pulley, the belt engaged to the first pulley. The system further comprises an inner member and an outer sheath secured to the belt and disposed about at least a portion of the inner member, and the system excludes a rack.

In another example, the disclosure is directed to a system for delivering an implantable endoprosthesis into a body vessel. The system comprises a handle assembly that comprises a housing, a rotatable member, a first pulley engaged to the rotatable member, a second pulley rotatably engaged to the housing, and a belt extending between the first pulley and the second pulley, the belt engaged to the first pulley. The system further comprises an inner member and an outer sheath secured to the belt and disposed about at least a portion of the inner member, and the system excludes a pull grip that extends proximally from the handle assembly.

In another example, this disclosure is directed to a system for delivering an implantable endoprosthesis into a body vessel. The system comprises a handle assembly that comprises a housing, a rotatable member, a first pulley engaged to the rotatable member, a second pulley rotatably engaged to the housing, and a belt extending between the first pulley and the second pulley, the belt engaged to the first pulley. The system further comprises an inner member, an outer sheath secured to the belt and disposed about at least a portion of the inner member, and a protuberance engaged to the belt and extending outwardly from the handle through a portion of the slot, where a force exerted by a user on the protuberance causes the belt to rotate about the first pulley and the second pulley, thereby causing the outer sheath to move proximally, where the system excludes a rack, and where the system excludes a pull grip that extends proximally from the handle assembly.

The details of one or more aspects of the disclosure are set forth in the accompanying drawings and the description below. Other features, objects, and advantages will be apparent from the description and drawings, and from the claims.

### BRIEF DESCRIPTION OF DRAWINGS

FIG. 1 is a broken, side view of an endoprosthesis delivery system, in accordance with this disclosure.

FIG. 2 is a cross-sectional view of a distal region of the endoprosthesis delivery system of FIG. 1, taken along line 2-2 in FIG. 1.

FIG. 3 is a side view of a handle assembly of the endoprosthesis delivery system of FIG. 1 in an operative configuration with the near side of its housing removed to expose certain interior components of the handle assembly.

FIG. 4 is a side view of the handle assembly of FIG. 3, shown with its housing attached.

### DETAILED DESCRIPTION

While this invention may be embodied in many different forms, there are described in detail herein specific examples of the invention. This description is an exemplification of the principles of the invention and is not intended to limit the invention to the particular examples illustrated.

This disclosure describes systems and techniques for delivering an implantable endoprosthesis, e.g., a stent, into a body lumen. The systems and techniques may be effective in reducing the length of a handle in a catheter system.

Many existing handles of catheter systems include rack and pinion designs for deploying the implantable endoprosthesis. As the pinion turns, e.g., via a thumbwheel engaged to the pinion, the rack moves linearly along the length of the handle. As the rack moves, a deployment sheath engaged to the rack retracts proximally, thereby exposing and deploying the distally-mounted implantable endoprosthesis.

The rack and pinion design of these existing systems results in long handle lengths, e.g., about 40 centimeters (cm). Because of the length of these existing handle designs, practitioners, e.g., doctors, often find these handles awkward to use. In addition, the long handles of existing systems require large amounts of packaging and take up considerable shelf space, e.g., at hospitals.

Existing catheter systems further include a pull grip engaged to the rack. The pull grip extends from the proximal end of the handle assembly. Many endoprostheses that are currently being deployed are too long for the rack alone to deploy the endoprosthesis. That is, at some point during the deployment of long endoprostheses, the rack can no longer engage the pinion. As a result, the practitioner must pull the pull grip proximally in order to fully deploy long endoprostheses. However, under the mistaken belief that the stent has been fully deployed once the rack is no longer engaged to the pinion, the practitioner may inadvertently pull the catheter system away from the deployment site before the stent has been fully deployed.

In accordance with various techniques of this disclosure, the length of the handle of the catheter system is significantly reduced over existing designs by removing the rack from the delivery system. As described in more detail below, a belt and pulley design replaces the rack and pinion design used in existing systems. The belt and pulley design described in this disclosure, which excludes a rack, allows the entire deployment mechanism to reside in the handle, thus significantly shortening the length of the handle, e.g., by about 50%. Reducing the length of the handle improves ease of use of the catheter system. In addition, the belt and pulley design simplifies deployment by excluding the pull grip of existing designs, thereby reducing the chance that a practitioner will inadvertently pull the catheter system away without fully deploying stent. Finally, reducing the length of the handle reduces both the amount of packaging and the amount of shelf space required.

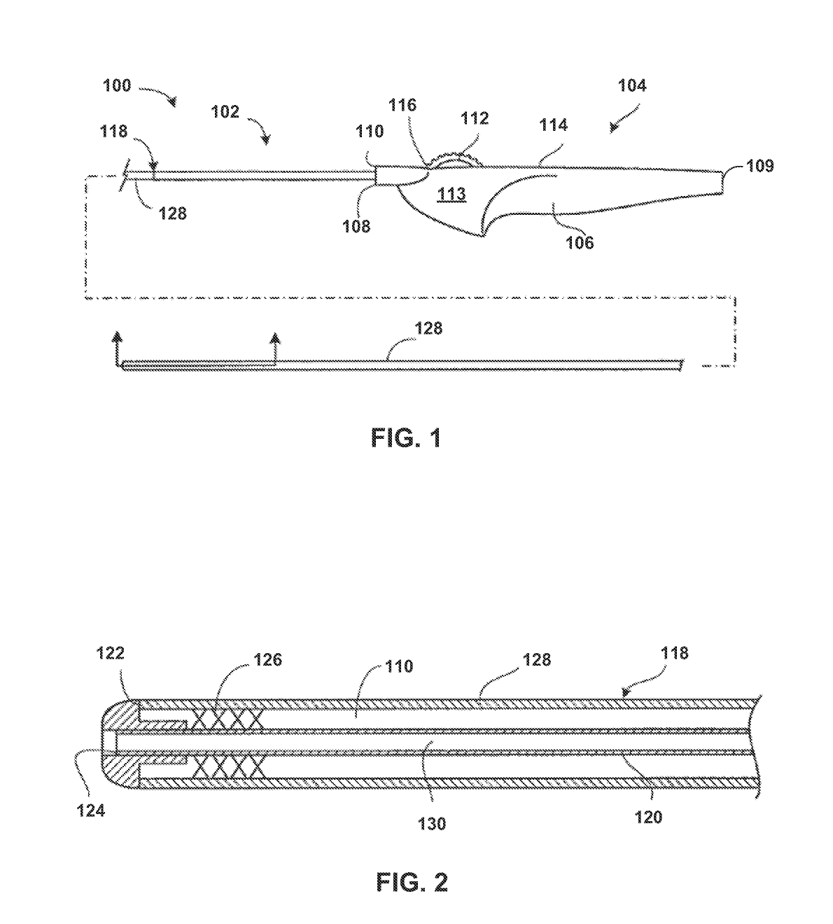

FIG. 1 is a broken, side view of an endoprosthesis delivery system. The techniques of this disclosure are applicable to numerous types of endoprostheses including, but not limited to, stents, grafts, and stent-grafts. For simplicity, this disclosure refers generally to these various endoprostheses as stents. FIG. 1 depicts stent delivery system 100 that includes catheter assembly 102 and handle assembly 104.

Handle assembly 104 includes housing 106 having distal end 108 and proximal end 109. Distal end 108 defines distal opening 110 that provides sufficient clearance for outer sheath 128 to pass through the opening and thus move axially through housing 106 without substantial interference. Outer sheath 128 may also be referred to as a deployment sheath.

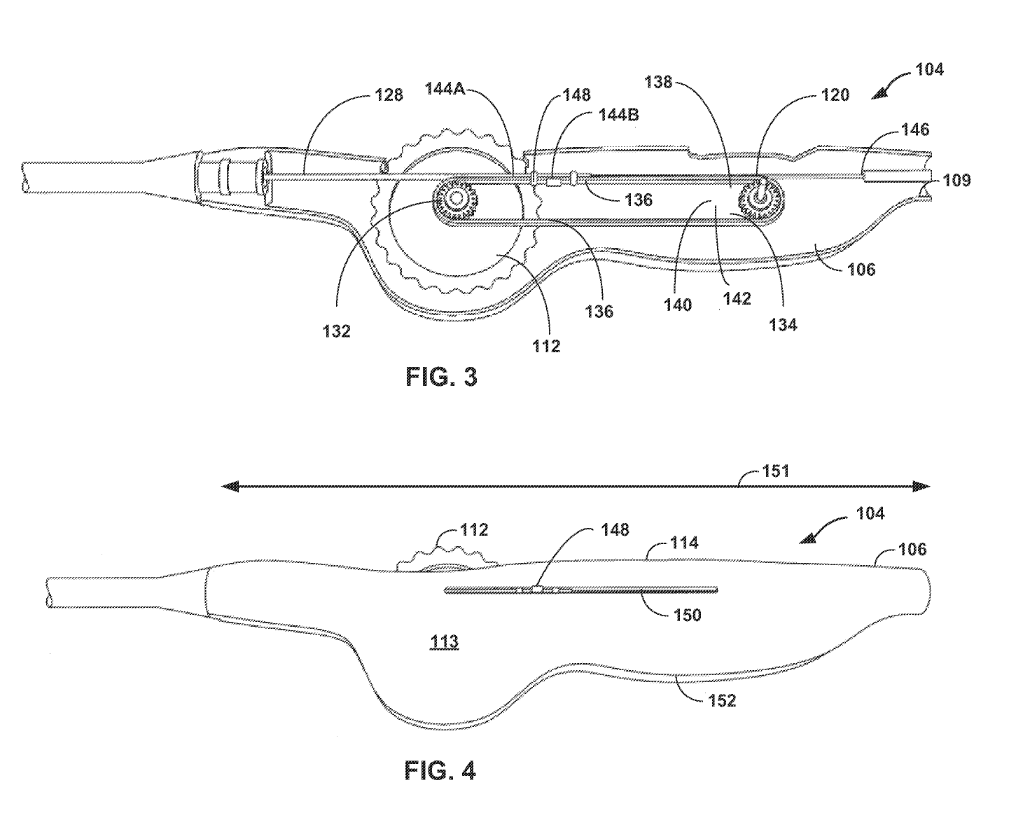

Handle assembly 104 further includes rotatable member 112, e.g., a thumbwheel, rotatably mounted to housing 106, as shown and described in more detail below with respect to FIG. 3. Housing 106 includes side wall 113 and top wall 114. Top wall 114 of housing 106 includes aperture 116 through which an upper portion of rotatable member 112 protrudes to allow rotatable member 112 to be rotated by the thumb of a user, which, in turn, retracts an outer sheath, e.g., outer sheath 128, to deploy a stent, e.g., stent 126 of FIG. 2. Stent 126 may be a self-expanding stent, a balloon-expandable stent, or a hybrid stent that is both self-expanding and balloon-expandable. As shown and described in more detail below with respect to FIG. 2, the belt and pulley design described in this disclosure allows the entire deployment mechanism to reside within handle assembly 104, thus significantly shortening the length of handle assembly 104.

FIG. 2 is a cross-sectional view of a distal region of the endoprosthesis delivery system of FIG. 1, taken along line 2-2 in FIG. 1. As seen in FIG. 2, catheter assembly 102 includes outer tubular assembly 118 and inner tubular member 120 (also referred to as simply an inner member) extending through guidewire lumen 130 formed by outer tubular assembly 118.

A self-expanding stent 126 is disposed between outer tubular assembly 118 and inner tubular member 120, near distal ends 122, 124 of outer tubular assembly 118 and inner tubular member 120. During use, a distal portion of catheter assembly 102 can be disposed within a body vessel (e.g., blood vessel) of a patient, and outer tubular assembly 118 can be retracted proximally relative to inner tubular member 120 to deploy stent 126 within the body vessel of the patient. In particular, outer sheath 128 of outer tubular assembly 118 can be retracted proximally toward handle assembly 104 in order to deploy stent 126.

Inner tubular member 120 forms guidewire lumen 130 that extends from the proximal end of inner tubular member 120 to the distal end of inner tubular member 120. A proximal end region of inner tubular member 120 extends into and is secured to housing 106 of handle assembly 104.

FIG. 3 is a side view of a handle assembly of the endoprosthesis delivery system of FIG. 1 in an operative configuration with the near side of its housing removed to expose certain interior components of the handle assembly. FIG. 3 depicts a belt and pulley design that replaces the rack and pinion design used in existing systems. As indicated above, the belt and pulley design shown in FIG. 3 allows the entire deployment mechanism to reside within handle assembly 104, thus significantly reducing the profile of the handle assembly by shortening its length.

As shown in FIG. 3, handle assembly 104 further includes first pulley 132 engaged to rotatable member 112, second pulley 134, and belt 136 extending between first pulley 132 and second pulley 134. Second pulley 134 is disposed about pin 138, which is secured to housing 106 of handle assembly 104, thereby allowing second pulley 134 to rotate freely. A force exerted by a user on rotatable member 112 causes first pulley 132 to rotate belt 136, thereby causing outer sheath 128 to move proximally. In some examples, one or both of first pulley 132 and second pulley 134 comprise a plurality of circumferentially spaced teeth 140 that extend radially from a peripheral surface 142 of the pulley. Teeth 140 may allow pulleys 132, 134 to grip belt 136 more securely. In other examples, one or both of first pulley 132 and second pulley 134 comprise ribbed, corrugated, or other uneven surfaces rather than teeth.

In some examples, belt 136 is comprised of a plastic material. In other examples, belt 136 is comprised of a metallic material. In some examples, belt 136 is a thread. In another example, belt 136 is a string or other braided filament. In one example, belt 136 is a chain, i.e., a series of links or rings fitted together to form a flexible member. In some examples, the chain is a plastic chain. In other examples, belt 136 is a metal chain.

In one example (not depicted), belt 136 comprises teeth spaced along the length of the belt, e.g., similar to that of a cable tie. These teeth may allow belt 136 to engage and grip pulleys 132, 134 more securely.

In contrast to existing designs that utilize a rack and pinion combination for transmission of force distal to proximal, the endoprothesis delivery system 100 of this disclosure transmits force through belt 136. As seen in FIG. 3, outer sheath 128 is attached directly to belt 136. Attaching outer sheath 128 directly to belt 136 improves ease of assembly because delivery system 100 is no longer indexed by the length of the rack, as is the case in existing designs.

Outer sheath 128 may be attached to belt 136 using various known techniques. For example, as depicted in FIG. 3, outer sheath 128 is bonded to belt 136 via two ties, namely ties 144A and 144B. In other examples, outer sheath 128 is bonded to belt 136 using an adhesive, using laser bonding, or through other securement means known to those of ordinary skill in the art. It should be noted that these securement means may be used in combination with one another, e.g., ties 144A and 144B in combination with an adhesive.

Handle assembly 104 further includes inner anchor 146 secured to the proximal end of inner tubular member 120 Inner anchor 146 abuts proximal end 109 of housing 106, thereby preventing inner tubular member 120 from moving proximally as outer sheath 128 is retracted proximally into handle assembly 104 over inner tubular member 120.

In some example configurations, handle assembly 104 further includes nub or protuberance 148 engaged to a portion of belt 136, for example. As shown and described in detail below with respect to FIG. 4, protuberance 148 extends outwardly through a portion of a slot defined by housing 106. Instead of using rotatable member 112 to retract outer sheath 128, a user can exert a force on protuberance 148, thereby causing belt 136 to rotate about first pulley 132 and second pulley 134 and to retract outer sheath 128 proximally.

FIG. 4 is a side view of the handle assembly of FIG. 3, shown with its housing attached. As seen in FIG. 4, protuberance 148 is engaged to a portion of belt 136 and extends outwardly from handle assembly 104 through a portion of a slot defined by housing 106. In particular, protuberance 148 extends outwardly through a portion of a slot defined by side wall 113 of housing 106. Slot 150 extends longitudinally along at least a portion of length 151 of handle assembly 104.

In other examples (not depicted), protuberance 148 may extend outwardly from handle assembly 104 through a portion of a slot defined by top wall 114, or bottom wall 152, of housing 106. In some examples, protuberance 148 may be engaged to a portion of outer sheath 128 in addition to or instead of belt 136. For example, protuberance 148 may be engaged to a portion of outer sheath 128 adjacent one of ties 144A, 144B.

As mentioned above, a user can exert a force on protuberance 148, rather than rotatable member 112, to retract outer sheath 128 to retract outer sheath 128 proximally. In some examples, using protuberance 148 instead of rotatable member 112 may retract outer sheath 128 more quickly.

The above disclosure is intended to be illustrative and not exhaustive. The description will suggest many variations and alternatives to those of ordinary skill in the art. All of these alternatives and variations are intended to be included within the scope of the attached claims. Those familiar with the art may recognize other equivalents to the specific embodiments described herein which equivalents are also intended to be encompassed by the claims attached hereto.

1. A system for delivering an implantable endoprosthesis into a body vessel, the system comprising:
a handle assembly comprising:
a housing;
a rotatable member;
a first pulley engaged to the rotatable member;
a second pulley rotatably engaged to the housing; and
a belt extending between the first pulley and the second pulley, the belt engaged to the first pulley;
an inner member; and
an outer sheath secured to the belt and disposed about at least a portion of the inner member,
wherein the system excludes a rack.

2. The system of claim 1, wherein a force exerted by a user on the rotatable member causes the first pulley to rotate the belt, thereby causing the outer sheath to move proximally.

3. The system of claim 1, wherein the outer sheath is retractable relative to the inner member.

4. The system of claim 1, wherein the belt comprises a braided filament.

5. The system of claim 1, wherein the belt has teeth that engage at least one of the first pulley and the second pulley.

6. The system of claim 1, wherein the belt is a chain.

7. The system of claim 6, wherein the chain is one of a plastic chain and a metal chain.

8. The system of claim 1, wherein at least one of the first pulley and the second pulley comprise a plurality of circumferentially spaced teeth that extend radially from a peripheral surface.

9. The system of claim 1, wherein the outer sheath and the inner member are configured so that the implantable endoprosthesis can be disposed between the outer sheath and the inner member.

10. The system of claim 1, wherein at least a portion of the outer sheath can be retracted into the handle assembly.

11. The system of claim 1, further comprising an implantable endoprosthesis disposed between the outer sheath and the inner member.

12. The system of claim 11, wherein the implantable endoprosthesis comprises a self-expanding stent.

13. The system of claim 1, wherein the handle defines a slot extending longitudinally along at least a portion of the length of the handle, the system further comprising:
a protuberance engaged to the belt and extending outwardly from the handle through a portion of the slot,
wherein a force exerted by a user on the protuberance causes the belt to rotate about the first pulley and the second pulley, thereby causing the outer sheath to move proximally.

14. The system of claim 13, wherein the protuberance extends outwardly from a side of the handle through a portion of the slot.

15. The system of claim 13, wherein the protuberance extends outwardly from a top of the handle through a portion of the slot.

16. A system for delivering an implantable endoprosthesis into a body vessel, the system comprising:
a handle assembly comprising:
a housing;
a rotatable member;
a first pulley engaged to the rotatable member;
a second pulley rotatably engaged to the housing; and
a belt extending between the first pulley and the second pulley, the belt engaged to the first pulley;
an inner member; and
an outer sheath secured to the belt and disposed about at least a portion of the inner member,
wherein the system excludes a pull grip that extends proximally from the handle assembly.

17. A system for delivering an implantable endoprosthesis into a body vessel, the system comprising:
a handle assembly comprising:
a housing;
a rotatable member;
a first pulley engaged to the rotatable member;
a second pulley rotatably engaged to the housing; and
a belt extending between the first pulley and the second pulley, the belt engaged to the first pulley;
an inner member;
an outer sheath secured to the belt and disposed about at least a portion of the inner member; and
a protuberance engaged to the belt and extending outwardly from the handle through a portion of the slot,
wherein a force exerted by a user on the protuberance causes the belt to rotate about the first pulley and the second pulley, thereby causing the outer sheath to move proximally,
wherein the system excludes a rack, and
wherein the system excludes a pull grip that extends proximally from the handle assembly.
